(19) United States
(12) Patent Application Publication
(10) Pub. No.: US 2025/0381729 A1
(43) Pub. Date: Dec. 18, 2025

(54) FDM PRINTED OBJECTS WITH HIGH-PERFORMANCE PHOTOCATALYTIC LAYERS

(71) Applicant: SIGNIFY HOLDING B.V., EINDHOVEN (NL)

(72) Inventors: TIES VAN BOMMEL, HORST (NL); RIFAT ATA MUSTAFA HIKMET, EINDHOVEN (NL)

(21) Appl. No.: 18/874,991

(22) PCT Filed: Jun. 13, 2023
(86) PCT No.: PCT/EP2023/065778

§ 371 Date: Dec. 13, 2024

(30) Foreign Application Priority Data
Jun. 17, 2022 (EP) 22179607.1

(51) Int. Cl. B29C 64/118; A61L 9/20; B01J 35/39; B29C 64/194; B29K 27/12; B29K 105/00; B33Y 10/00; B33Y 40/00; B33Y 70/10; B33Y 80/00; D01F 1/10; D01F 8/10

(52) U.S. Cl. CPC B29C 64/118; A61L 9/205; B01J 35/39; B29C 64/194; B33Y 10/00; B33Y 40/00; B33Y 70/10; B33Y 80/00; D01F 1/10; D01F 8/10; A61L 2209/12; A61L 2209/134; B29K 2027/12; B29K 2105/0014; D10B 2321/04; D10B 2505/00

(57) ABSTRACT

The invention provides a method for producing a 3D item (1) by means of fused deposition modelling, the method comprising: a 3D printing stage comprising layer-wise depositing 3D printable material (201), to provide the 3D item (1) comprising 3D printed material (202), wherein the 3D item comprises layers (322) of 3D printed material, wherein the 3D printable material comprises a thermoplastic material (401) and a photocatalytic material (409) wherein during at least part of the 3D printing stage the method comprises producing pores (423) in the 3D printable material.

### FIELD OF THE INVENTION

The invention relates to a method for manufacturing a 3D (printed) item. Further, the invention relates to a filament for producing such item (with such method). The invention also relates to the 3D (printed) item obtainable with such method. Further, the invention relates to a radiation generating system including such 3D (printed) item. Yet further, the invention also relates to a method for treating a gas (with such system).

### BACKGROUND OF THE INVENTION

The use of photocatalysts for sterilization is known in the art. For instance, US20200101440A1 describes a monolithic composite photocatalyst comprising a photoactive nanocrystal component, and a non-photoactive porous support. Photocatalytic fluid purification systems that contact an impurity-containing fluid with the subject monolithic composite photocatalysts are also described in US20200101440A1.

### SUMMARY OF THE INVENTION

UV light has been used for disinfection for over 100 years. Wavelengths between about 190 nm and 300 nm may be strongly absorbed by nucleic acids, which may result in defects in an organism's genome. This may be desired for inactivating (killing), bacteria and viruses, but may also have undesired side effects for humans. Therefore, the selection of wavelength of radiation, intensity of radiation and duration of irradiation may be limited in environments where people may reside such as offices, public transport, cinema's, restaurants, shops, etc., thus limiting the disinfection capacity. Especially in such environments, additional measures of disinfection may be advantageous to prevent the spread of bacteria and viruses such as influenza or novel (corona) viruses like COVID-19, SARS and MERS.

It appears desirable to produce systems, that provide alternative ways for air treatment, such as disinfection. Further, existing systems for disinfection may not easily be implemented in existing infrastructure, such as in existing buildings like offices, hospitality areas, etc. and/or may not easily be able to serve larger spaces. This may again increase the risk of contamination. Further, incorporation in HVAC systems may not lead to desirable effects and appears to be relatively complex. Further, existing systems may not be efficient, or may be relatively bulky, and may also not easily be incorporated in functional devices, such as e.g. luminaires.

Other disinfection systems may use one or more anti-microbial and/or anti-viral means to disinfect a space or an object. Examples of such means may be chemical agents which may raise concerns. For instance, the chemical agents may also be harmful for people and pets.

In embodiments, the disinfecting light, may especially comprise ultraviolet (UV) radiation (and/or optionally violet radiation), i.e., the light may comprise a wavelength selected from the ultraviolet wavelength range (and/or optionally the violet wavelength range). However, other wavelengths are herein not excluded. The ultraviolet wavelength range is defined as light in a wavelength range from 100 to 380 nm and can be divided into different types of UV light/UV wavelength ranges (Table 1). Different UV wavelengths of radiation may have different properties and thus may have different compatibility with human presence and may have different effects when used for disinfection (Table 1).

TABLE 1
Properties of different types of UV, violet, and NIR wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Near | NIR | 780-950 | + | +/− | +++ | | |
| Infrared | | | | | | | |
| (deep red) | | | | | | | |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near | Near | 230-280 | ++ | ++ | − | | |
| ultraviolet | UV-C | | | | | | |
| C | | | | | | | |
| Far ultra- | Far | 190-230 | +++ | +++ | + | | +/− |
| violet C | UV-C | | | | | | |
| Extreme | Extreme | 100-190 | +++ | +++ | | | + |
| ultraviolet | UV-C | | | | | | |
| C | | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may inactivate (kill) bacteria, but may be less effective in inactivating (killing) viruses. UV-B may be (relatively) safe when a low dose (i.e. low exposure time and/or low intensity) is used, may inactivate (kill) bacteria, and may be moderately effective in inactivating (killing) viruses. UV-B may also have the additional benefit that it can be used effectively in the production of vitamin D in a skin of a person or animal. Near UV-C may be relatively unsafe, but may effectively inactivating, especially kill bacteria and viruses. Far UV-C may also be effective in inactivating (killing) bacteria and viruses, but may be (relatively to other UV-C wavelength ranges) (rather) safe. Far-UV light may generate some ozone which may be harmful for human beings and animals. Extreme UV-C may also be effective in inactivating (killing) bacteria and viruses, but may be relatively unsafe. Extreme UV-C may generate ozone which may be undesired when exposed to human beings or animals. In some application ozone may be desired and may contribute to disinfection, but then its shielding from humans and animals may be desired. Hence, in the table “+” for ozone production especially implies that ozone is produced which may be useful for disinfection applications, but may be harmful for humans/animals when they are exposed to it. Hence, in many applications this “+” may actually be undesired while in others, it may be desired. The types of light indicated in above table may in embodiments be used to sanitize air and/or surfaces.

The terms “inactivating” and “killing” with respect to a virus may herein especially refer to damaging the virus in such a way that the virus can no longer infect and/or reproduce in a host cell, i.e., the virus may be (essentially) harmless after inactivation or killing.

Hence, in embodiments, the light may comprise a wavelength in the UV-A range. In further embodiments, the light may comprise a wavelength in the UV-B range. In further embodiments, the light may comprise a wavelength in the Near UV-C range. In further embodiments, the light may comprise a wavelength in the Far UV-C range. In further embodiments, the light may comprise a wavelength in the extreme UV-C range. The Near UV-C, the Far UV-C and the extreme UV-C ranges may herein also collectively be referred to as the UV-C range. Hence, in embodiments, the light may comprise a wavelength in the UV-C range. In other embodiments, the light may comprise violet radiation.

Hence, light or radiation described herein may also be indicated as disinfection light.

It appears desirable to protect people from the spread of bacteria and viruses such as influenza or against the outbreak of novel (corona) viruses like COVID-19, SARS and MERS. Ultraviolet (UV) light has been used for disinfection for over 100 years, however, depending on the wavelength used, the UV light may not kill viruses, only bacteria. Hence, there is a desire to provide optimized devices. Yet, it also appears desirable to produce items or item comprising apparatus, such as luminaires or other radiation generating systems, which may especially be designed for specific applications and/or allow a relatively free shaping of the apparatus.

Hence, it is an aspect of the invention to provide an alternative method for making an item that can e.g. be used in the treatment of air and/or an alternative item (e.g. for such use) as such, which may preferably further at least partly obviate one or more of above-described drawbacks. The present invention may have as object to overcome or ameliorate at least one of the disadvantages of the prior art, or to provide a useful alternative.

Amongst others, in embodiments, herein a combination of UV light and photocatalysis for improved disinfection is proposed. Yet, in embodiments an optimized 3D printing process is proposed, which may especially be applied to provide a support for the photocatalyst.

Within the next 10-20 years, digital fabrication will increasingly transform the nature of global manufacturing. One of the aspects of digital fabrication is 3D printing. Currently, many different techniques have been developed in order to produce various 3D printed objects using various materials such as ceramics, metals, and polymers. 3D printing can also be used in producing molds which can then be used for replicating objects.

For the purpose of making molds, the use of polyjet technique has been suggested. This technique makes use of layer by layer deposition of photo-polymerizable material which is cured after each deposition to form a solid structure. While this technique produces smooth surfaces the photo curable materials are not very stable, and they also have relatively low thermal conductivity to be useful for injection molding applications.

The most widely used additive manufacturing technology is the process known as Fused Deposition Modeling (FDM). Fused deposition modeling (FDM) is an additive manufacturing technology commonly used for modeling, prototyping, and production applications. FDM works on an “additive” principle by laying down material in layers; a plastic filament or metal wire is unwound from a coil and supplies material to produce a part. Possibly, (for thermoplastics for example) the filament is melted and extruded before being laid down. FDM is a rapid prototyping technology. Other terms for FDM are “fused filament fabrication” (FFF) or “filament 3D printing” (FDP), which are considered to be equivalent to FDM. In general, FDM printers use a thermoplastic filament, which is heated to its melting point and then extruded, layer by layer, (or in fact filament after filament) to create a three-dimensional object. FDM printers are relatively fast, low cost and can be used for printing complicated 3D objects. Such printers are used in printing various shapes using various polymers. The technique is also being further developed in the production of LED luminaires and lighting solutions.

Hence, in a first aspect the invention provides a method for producing a 3D item by means of fused deposition modelling. Especially, the method may comprise (a 3D printing stage comprising) layer-wise depositing 3D printable material, to provide the 3D item comprising 3D printed material. Especially, the 3D item may comprise layers of 3D printed material. In embodiments, the 3D printable material may comprise a thermoplastic material. In embodiments, the 3D printable material may further comprise a photocatalytic material. Especially, (during at least part of the 3D printing stage) the method may comprise producing pores in the 3D printable material. Therefore, in specific embodiments the invention provides a method for producing a 3D item by means of fused deposition modelling, the method comprising: a 3D printing stage comprising layer-wise depositing 3D printable material, to provide the 3D item comprising 3D printed material, wherein the 3D item comprises layers of 3D printed material, wherein the 3D printable material comprises a thermoplastic material and a photocatalytic material wherein during at least part of the 3D printing stage the method comprises producing pores in the 3D printable material.

This may allow a relatively simple 3D printing method to provide FDM printed objects with high-performance photocatalytic layers. Further, in this way, it may be possible to create a radiation generating system which may provide improved disinfection compared to state of the art UV radiation systems. Yet, such system may comprise a tailor made element, that may be shaped to the desired end user application. The combination of the photocatalyst, UV radiation, and an increased surface area may be desirable for killing viruses amongst other pathogens. Disinfection performance may be improved by combining UV light with a photocatalyst. It may also enable using material combinations resistant to UV and placing photocatalytic layers where they may work in a more efficient way.

As indicated above the invention may provide a method for producing a 3D printed item by means of fused deposition modelling. The 3D printed item may comprise one or more layers of 3D printed material. Especially, the 3D printed item may comprise a plurality of layers of 3D printed material. One or more of these layers may comprise at least a part (“layer part”) with a 3D printed material that is porous and comprises a photocatalyst.

Especially, the method may comprise layer-wise depositing a 3D printable material comprising the photocatalyst while generating porosity. Hence, a stack of layers may be provided. At least part of one of the layers, especially a part defined along a length axis of such layer, may thus be porous and comprise a photocatalyst. Note that other parts may have different compositions, and/or may not be porous and/or may not comprise the photocatalyst.

Photocatalysis for air purification may be based on the absorbance of radiation of suitable wavelengths, such as violet and/or UV light, by a photocatalytic material, resulting in the formation of reactive oxygen species (ROS). Such reactive oxygen species can decompose air pollutants and inactivate pathogens.

In embodiments, the photocatalytic material may include one or more of ZnO, ZnS, CdS, SrO₂, WO₃ and Fe—TiO₂. In embodiments, the photocatalytic material may comprise TiO₂. In specific embodiments, the photocatalytic material may comprises clusters of TiO₂ particles, especially clusters of >1000 TiO₂ particles. In embodiments, the photocatalytic material may comprise anatase (TiO₂). Photocatalytic particles may also comprise nano fibers. In embodiments, the photocatalytic particles may also comprise metals such as Au, Pt, and Pd attached on TiO₂ nanoparticles. In specific embodiments, the photocatalytic particles may comprise co-doped TiO₂ particles with N and W. In this way, the photocatalytic particles may show photo activity when irradiated at longer wavelengths in the visible range extending to 440 nm and even beyond.

As photocatalysis may especially occur at the interface of the photocatalytic material and air, increasing the surface area of contact may improve the performance of the photocatalytic layer. In embodiments, the surface area of contact may be increased by including pores in the 3D printed material. Hence, in embodiments, the method comprises producing pores in the 3D printable material.

In embodiments, pores may be introduced in the 3D printable material by incorporating a gas during the processing. For example, air may be incorporated into the melted thermoplastic material. In embodiments, this may be done via a core-nozzle of a core-shell nozzle. Additionally or alternatively, in embodiments a gas may be incorporated into the melted thermoplastic material in the printer head. Especially, the gas and the melted thermoplastic material may be mixed thoroughly, which may result in gas bubble formation, which may form the pores. As the melted thermoplastic material may be relatively viscous, such bubbles or pores may especially remain present during the extrusion and solidification of the melted thermoplastic material into solidified 3D printed material. In embodiments, a shell nozzle may be in communication with a part through which the core material is extruded. In this way, air, or another gas, may be introduced in the core material. For producing porous printed material it may also be possible to use solvents which start boiling at the printing temperature. Such solvents may be brought into the filaments used for printing during the production of the filament or soaking such a filament in a solvent before printing. It may also be possible to include molecules into the filament which disintegrate during printing and produce gases which leads to formation of pores.

Additionally or alternatively, the 3D printable material may in embodiments further comprises a pore forming material. Especially, during at least part of the 3D printing stage the method may comprise producing pores by conversion of the pore forming material. Hence, in specific embodiments the 3D printable material further comprises a pore forming material, wherein during at least part of the 3D printing stage the method comprises producing pores by conversion of the pore forming material. Hence, the 3D printable material like a filament of 3D printable or pellets of 3D printable material may comprise a thermoplastic material with pore forming material embedded therein. Alternatively or additionally, the pore forming material may be added to a printer head and mixed in the printer head with the thermoplastic material.

In embodiments, the pore forming material may comprise a liquid such as one or more of water, ethanol, methanol, isopropanol (or other propanol), n-hexane, cyclohexane, 4-dioxane, acetone, chloroform, dichloromethane, tetrahydrofuran, N,N-dimethylformamide, ethyl acetate, hexafluoroisopropanol, and hexafluoroacetone. The liquid pore forming material may be converted into a gas and in this way form pores in the 3D printable material. This conversion may take place during heating of the 3D printable material in a printer head, especially in a nozzle. Such expanding solvent or gases may expand in the nozzle and formed bubbles may burst as they leave the nozzle exposing the particle surfaces. The pore forming material may especially comprise one or more of ethanol and propanol. Alternatively or additionally, the pore forming material may especially comprise water.

Additionally or alternatively, the pore forming material may comprise particles comprising gas bubbles, such as porous particles. The gas (e.g. air, nitrogen, etc. . . . ) that is located inside the porous particles whilst being encapsulated within the printable material may expand during extrusion (because of the increased temperature in the nozzle) and therefore produce (larger) pores in the 3D printed material. Since the pores remain located around the embedded porous particles, the position of the pores can be controlled by controlling the position of the inorganic particles. As the size of the pores in the 3D printed material may depend upon the size of the pores in the porous particles, the porosity of the 3D printed material may also be controlled by the type of inorganic particles used.

In embodiments, the porosity of the porous particles may be in the range 5-80 vol. %, such as 20-60 vol. %. The porosity of the porous particles may determine the amount of gas or liquid that can expand. Porous particles with low porosity cannot produce large enough voids and if the porosity of the porous particles is too high, then the porous particles may be mechanically too weak, and they may break up into small pieces. The porosity may be determined via a direct method, such as especially determining the bulk volume of the porous sample, and then determining the volume of the skeletal material with no pores (pore volume=total volume−material volume). Alternatively, the porosity may be determined via an optical method, such as especially determining the area of the material versus the area of the pores visible under the microscope. The “areal” and “volumetric” porosities are essentially equal for porous media with random structure. Especially, an optical method may be applied.

Similarly, the pore size may at least partly determine the amount of gas or liquid that can expand and may also at least partly determine the strength of the particles. In embodiments, the inorganic particles have an average pore size in the range of 10-100 μm. The pore size may be determined via an optical method, such as especially measuring the diameter of the pores visible under the microscope. Alternatively, the porosity may be determined using especially mercury pressure porosimetry. Alternatively, especially X-ray refraction may be applied.

In embodiments, the porous particles may comprise inorganic particles. Many inorganic materials may be suitable for the porous inorganic particles, especially metal oxide particles appear to be favorable. In embodiments, the porous inorganic particles comprise porous glass particles. Herein, the term “metal oxide” may refer to MO based systems, but also to borates, silicates, phosphates, etc. Additionally or alternatively, the porous particles may comprise polymeric particles. In specific embodiments, the porous particles may comprise thermoplastic material. Especially, in embodiments the porous particles may comprise the same thermoplastic material as the thermoplastic material of the 3D printable material (and the 3D printed material). Alternatively, the porous particles may comprise a thermoplastic material different from the thermoplastic material of the 3D printable material. Thermoplastic materials are further described below.

In specific embodiments, the porous particles may comprise photocatalytic material. In this way, the formed pores may be especially in the proximity of the photocatalytic material. As indicated above, photocatalysis may especially occur at the interface of the photocatalytic material and air, having pores near the photocatalytic material may further increase the performance of the photocatalytic layers. Hence, in embodiments the photocatalyst may be provided as pore forming particle. In embodiments, the photocatalyst may comprise porous particles with a metal and/or metal oxide deposited thereon and therein (i.e. in the porous), such as a porous particle that is a support for TiO₂. Hence, in embodiments the phrase “a photocatalytic material, and a pore forming material”, and similar phrases may also refer to a porous material comprising photocatalytic material.

Additionally or alternatively, conversion of the pore forming material may comprise a chemical reaction. Especially, the chemical reaction may in embodiments comprise a gas forming reaction. For example, the production of carbon dioxide by reaction of an acid with a carbonate. However, other chemical reactions may also be possible. Alternatively or additionally, conversion of the pore forming material may comprise a decomposition reaction.

Conversion of the pore forming material may thus in embodiments comprise one or more of a phase transition of at least part of the pore forming material, expansion of at least part of the pore forming material or of a gas enclosed by the pore forming material, and chemical conversion of at least part of the pore forming material.

Especially, the conversion of the pore forming material to provide the pores may be induced by heat, though other methods are herein not excluded. Hence, in specific embodiments, the pore forming material may be heated. In alternative embodiments, UV light may be used for conversion of the pore forming material (optionally in combination with heat).

Especially, heating may be executed with a 3D printing apparatus. Hence, in embodiments the method may comprise using a 3D printing apparatus. Especially, the 3D printing apparatus may comprise a printer nozzle. Heat may be provided to the 3D printable material to induce pore formation in the nozzle as the nozzle may be heated anyhow. Hence, in embodiments a printer head (of a 3D printing apparatus) may comprise a heating element for heating the 3D printable material and to induce pore formation (during at least part of the 3D printing stage).

In specific embodiments, the pore forming material may comprise a material having a boiling point T_{b}. Especially, the 3D printing stage may comprise heating the pore forming material in the printer nozzle. Especially, the nozzle temperature T₁ may be above the boiling point T_{b}, especially when the pore forming material may comprise a material having a boiling point T_{b}. In embodiments, Tₙ−T_{b}≥2° C., such as Tₙ−T_{b}>10° C., especially Tₙ−T_{b}≥20° C. In specific embodiments, 30° C.≤T_{b}≤Tₙ, such as 50° C.≤T_{b}≤Tₙ, especially 100° C.≤T_{b}<Tₙ, like 200° C.≤T_{b}≤Tₙ. Hence, in specific embodiments the method may comprise using a 3D printing apparatus, wherein the 3D printing apparatus comprises a printer nozzle, wherein the pore forming material comprises a material having a boiling point T_{b}, wherein the 3D printing stage comprises heating the pore forming material in the printer nozzle wherein the printer nozzle has a nozzle temperature Tₙ, wherein 50° C.≤T_{b}≤Tₙ. Hence, especially heating the pore forming material in the printer nozzle may thus imply heating the pore forming material comprise 3D printable material in the printer nozzle.

Especially, in embodiments the pore forming material may comprise a liquid at room temperature that boils at a temperature selected from the range of 30-500° C., such as 50-350° C., like 75-350° C., especially 150-300° C.

Additionally or alternatively, the pore forming material may comprise a foaming agent. In embodiments, the pore forming material may comprise an inorganic foaming agent, such as sodium bicarbonate, ammonium carbonate, ammonium bicarbonate, and calcium azide. In alternative embodiments, the pore forming material may comprise an organic foaming agent, such as azodicarbonamide, hydrazocarbonamide, benzenesulfonyl hydrazide, dinitrosopentamethylene tetramine, toluenesulfonyl hydrazide, p,p′-oxybis(benzenesulfonylhydrazide), azobisisobutyronitrile, and barium azodicarboxylate.

Hence, in specific embodiments, the pore forming material comprises one or more of (i) a liquid at room temperature that boils at a temperature selected from the range of 75-350° C. and (ii) a foaming agent. As indicated above, the pore forming material may in embodiments comprise water or alcohol (or a combination thereof).

Would a gas be formed not by boiling, but by a chemical reaction or by decomposition, then in specific embodiments instead of a boiling temperature, a reaction temperature or a decomposition temperature may be chosen (and then T_{b} may be interpreted as such).

In embodiments, the method may further comprise selecting one or more of the pore forming material, 3D printable material, and the 3D printing conditions such that the 3D printed material has a pore volume selected from the range of 2-80 vol. %, like 5-70 vol. %, such as 10-60 vol. %, like 10-50 vol. %, especially 20-50 vol. %. Hence, in specific embodiments, the 3D printing stage may comprise selecting the 3D printable material, and the 3D printing conditions such that the 3D printed material has a pore volume selected from the range of 10-50 vol. %. Additionally or alternatively, in specific embodiments, the 3D printing stage comprises selecting the pore forming material, the 3D printable material, and the 3D printing conditions such that the 3D printed material may have a pore volume selected from the range of 10-50 vol. %. For instance, the 3D printing conditions may refer to one or more of relative amounts of materials, temperature, 3D printing speed, etc.

As photocatalysis may essentially only occur at the interface of the photocatalytic material and air (or another gas), the appearance of the photocatalytic material may influence the amount of photocatalysis that may occur. In embodiments, the 3D printable material may comprise particles comprising the photocatalytic material. The particles may at least partly be randomly distributed through the printable material.

As indicated above, the pore forming material may in embodiments comprise porous particles. Also indicated above is that the porous particles may especially comprise photocatalytic material. Hence, in specific embodiments, the photocatalytic particles may be the porous particles. Especially, the photocatalytic particles may comprise pores.

The shape of the particles may influence the efficiency of the photocatalysis. In embodiments, the particles may be spherical, cubic, prismoid, flakes, or irregularly shaped. Particle sizes are especially may be selected such that the particles can pass the printer nozzle without clog formation. Particles having a relatively large surface area may be more efficient than e.g. spherical particles. Further, non-spherical particles may also have a chance to protrude from the 3D printed material, e.g. into pores created in the 3D printed material with the herein described method.

Especially, particles may have the shape of flakes. In embodiments the 3D printable material may comprise photocatalytic material comprises flakes. Hence, the 3D printable material may in embodiments comprises flakes comprising the photocatalytic material. In embodiments, the flakes may have flake dimensions defined by smallest rectangular prisms circumscribing the respective flakes, wherein such rectangular prism has a length L1, a width L2 and a height L3. In embodiments L1≥L2≥L3. In embodiments, length L1 is selected from the range of 50-2000 μm, especially selected from the range of 100-2000 μm, especially selected from the range of 250-1500 μm, more especially selected from the range of 500-1000 μm. Such rectangular prism has a first aspect ratio AR1=L1/L3, and a second aspect ratio AR2=L2/L3. In embodiments, the aspect ratios AR1 and AR2 are individually selected from the range of 1-10000, especially in the range of 2-1000, more especially in the range of 5-500, such as greater than 5. Hence, in specific embodiments, the 3D printable material comprises flakes comprising the photocatalytic material, wherein the flakes have flake dimensions defined by smallest rectangular prisms circumscribing the respective flakes, wherein such rectangular prism has a length L1, a width L2 and a height L3, wherein the length L1 is selected from the range of 50-2000 μm, wherein a first aspect ratio is AR1=L1/L3, wherein a second aspect ratio is AR2=L2/L3, wherein the aspect ratios AR1 and AR2 are individually selected from the range of 1-10000.

In embodiments, the particles may have dimensions selected from the range of 10 nm-2000 μm, such as selected from the range of 50 nm-2000 μm, like e.g. selected from the range of 0.1-2000 μm, such as 0.1-1000 μm, like 0.1-500 μm, like selected from the range of 1-200 μm.

In embodiments, the particles may have equivalent spherical diameters selected from the range of 10 nm-2000 μm, such as selected from the range of 50 nm-2000 μm, like e.g. selected from the range of 0.1-2000 μm, such as 0.1-1000 μm, like 0.1-500 μm, like selected from the range of 1-200 μm.

The equivalent spherical diameter (or ESD) of an (irregularly) shaped object is the diameter of a sphere of equivalent volume. Hence, the equivalent spherical diameter (ESD) of a cube with a side a is

$$2 * a * \sqrt[3]{3 / ( 4 * π )} .$$

Would a sphere in an xyz-coordinate system with a diameter D be distorted to any other shape (in the xyz-plane), without changing the volume, than the equivalent spherical diameter of that shape would be D.

Particle sizes (particles in general, hence including particles comprising the photocatalytic material and porous particles) may be determined with methods known in the art, like one or more of optical microscopy, SEM and TEM. Dimensions may be number averaged, as known in the art. Further, the aspect ratios indicated above may refer to a plurality of particles having different aspect ratios. Hence, the particles may be substantially identical, but the particles may also mutually differ, such as two or more subsets of particles, wherein within the subsets the particles are substantially identical. The particles may have a unimodal particle size distribution or a polymodal size distribution.

The particles, such as comprising the photocatalytic material (such as the flakes comprising the photocatalytic material), may thus mutually differ. For instance, the particles may have a distribution of the sizes of one or more of the particle length, the particle height, and an intermediate length. Therefore, in embodiments in average, the particles will have dimensions as described herein. For instance, at least 50 wt % of the particles may comply with the herein indicated dimensions (including ratios), such as at least 75 wt %, like at least 85 wt %. In alternative embodiments, at least 50% of the total number of particles may comply with the herein indicated dimensions (including ratios), such as at least 75%, like at least 85%.

In embodiments, the flakes comprising the photocatalytic material may be obtained by fragmenting a photocatalytic layer into flakes to obtain the flakes. The photocatalytic layer may in embodiments comprise a coating of the photocatalytic material on a carrier material. Hence, fragmenting the photocatalytic layer may comprise fragmenting the carrier material and the coating of the photocatalytic material. Additionally or alternatively, the photocatalytic layer may comprise a layer element of the photocatalytic material, like a thin plate of the photocatalytic material.

The contact surface area of the photocatalytic material and air may in embodiments be increased by having particles or flakes comprising the photocatalytic material protrude from the 3D printed material. Additionally or alternatively, post processing techniques may expose particles comprising the photocatalytic material embedded in the thermoplastic material so they may protrude from the thermoplastic material. Such post processing technique may include removing part of the 3D printed material, especially removing part of the thermoplastic material. Hence, in embodiments, the method may comprise dissolving a part of the 3D printed material (especially the thermoplastic material) by exposing at least part of the 3D printed material to a solvent for the 3D printed material. In this way, especially an outer section of the thermoplastic material (and hence an outer section of the 3D item) may be removed. This may result in more particles comprising the photocatalytic material protruding from the (remaining) thermoplastic material which may result in a larger surface area of photocatalytic material in contact with air. Again, a larger surface area of photocatalytic material in contact with air may provide photocatalytic layers with improved performance.

As indicated above, the invention may comprise embodiments wherein the 3D printable material may comprise particles, such as particles comprising the photocatalytic material and/or porous particles. In such embodiments, relative to a total volume of the 3D printable material, the volume percentage of the particulate material may be selected from the range of 15-60 vol. %. Consequently, relative to a total volume of the 3D printed material, the volume percentage of the particulate material is selected from the range of 15-60 vol. %. Hence, relative to a total volume of the 3D printable material or the 3D printed material, the volume percentage of the particulate material (relative to the 3D printable material or the 3D printed material, respectively) is selected from the range of 15-60 vol. %. Smaller values than about 15 vol. %, such as smaller than 10 vol. %, may not have the desired structural effect, larger values than about 70 vol. %, such as larger than about 60 vol. % may become very difficult to print. In embodiments, the remainder of the volume may be polymeric material, and optionally other fillers (see also below).

When referring to the concentration of the particles during deposition of the filaments or after deposition of the filaments, the concentration especially refers to at least part of such filament, or at least part of the deposited layer. Hence, the concentration of the particles may vary over the length of the filament or may vary over a length of a layer, or differ between layers. It is even possible that there are layers without particles and layers with particles. The smallest (integral) volume for which the concentration applies is especially at least 1 cm³, such as at least 2 cm³, like at least 5 cm³. Of course, this may be a relative extended volume, as the height and width of the layers are in general relatively small.

In embodiments, the 3D item that may be produced by the method of this invention, may be exposed to UV light. Especially, the photocatalytic material may be exposed to UV light. As a consequence, the 3D printed material may also be exposed to UV light. UV light may degraded some types of 3D printed materials (and 3D printable materials). To prevent the 3D printed material from degrading by the UV light, for instance fluoropolymers may be included in the 3D printable material. Hence, in specific embodiments the 3D printable material comprises one or more fluoropolymers.

In embodiments, fluoropolymers may be included as small particles in the (melt processable) 3D printable material. The fluoropolymer particles may especially comprise polytetrafluorethylene (PTFE). Additionally or alternatively, the 3D printable material may comprise a melt processable fluoropolymer. In embodiments, the fluoropolymers may comprise copolymers of tetrafluoroethylene (TFE) with one or more other perfluorinated, partially fluorinated or non-fluorinated comonomers.

In embodiments, the fluoropolymers may comprise copolymers of TFE and perfluorinated alkyl or allyl ethers, which are known in the art as PFA's (perfluorinated alkoxy polymers). Additionally or alternatively, the fluoropolymers may comprise copolymers of TFE and hexafluoropropylene (HFP) with or without other perfluorinated comonomers, which are known in the art as FEP's (fluorinated ethylene propylene). Additionally or alternatively, the fluoropolymers may comprise copolymers of TFE, HFP and vinylidenefluoride (VDF), which are known in the art as THV. In embodiments, other types of melt-processable fluoropolymers may be based on vinylidenefluoride homo- or copolymers, known in the art as PVDF. Additionally or alternatively, the fluoropolymers may comprise fluorinated ethylenic-cyclo oxyaliphatic substituted ethylenic copolymer, which is a family of amorphous fluoropolymers based on copolymers of 2,2-bistrifluoromethyl-4,5-difluoro-1,3-dioxole (PDD).

As indicated above, the photocatalytic performance of the 3D item may depend on the contact surface area of the photocatalytic material and air. Photocatalytic material that is fully embedded in 3D printed material may not substantially contribute to the photocatalytic performance and hence disinfection properties of the 3D item. Therefore, in embodiments, photocatalytic material may desirably be present in higher concentrations near the outer surface of the layers. This may in embodiments be achieved by using a core-shell filament for 3D printing. Hence, in embodiments, the 3D printing stage may comprise: layer-wise depositing a filament comprising the 3D printable material. Especially, the filament may comprise a core-shell filament comprising (i) a core and (ii) a shell, wherein the shell at least partly encloses the core. Especially, the core and (ii) a shell may comprise thermoplastic material. In specific embodiments, a second concentration c2 of photocatalytic material comprised by the shell may be larger than a first concentration c1 of photocatalytic material in the core. In specific embodiments, c1/c2≤0.9, such as c1/c2≤0.8, like c1/c2≤0.5. In embodiments, no photocatalytic material may be present in the core. Thus in embodiments c1 may be zero.

Hence, in specific embodiments, the 3D printing stage comprises: layer-wise depositing a filament comprising the 3D printable material, wherein the filament comprises a core-shell filament comprising (i) a core and (ii) a shell, wherein the shell at least partly encloses the core, wherein the core and (ii) a shell comprise thermoplastic material; wherein a second concentration of photocatalytic material comprised by the shell is larger than a first concentration of photocatalytic material in the core.

In alternative embodiments, higher concentrations of the photocatalytic material near the outer surface of the layers may be achieved using 3D printer comprising a core-shell nozzle. Therefore, in embodiments the method may comprise using a fused deposition modeling 3D printer (“3D printer”), wherein in specific embodiments the fused deposition modeling 3D printer comprises a first printer nozzle and a second printer nozzle, (the second printer nozzle) at least partly enclosing the first printer nozzle, wherein the method may comprise one or more of (i) feeding the 3D printable core material to the first printer nozzle and (ii) feeding the 3D printable shell material to the second printer nozzle. In specific embodiments, a second concentration c2 of photocatalytic material comprised by the 3D printable shell material may be larger than a first concentration c1 of photocatalytic material in the 3D printable core material. In specific embodiments, c1/c2≤0.9, such as c1/c2≤0.8, like c1/c2≤0.5. The fused deposition modeling 3D printer may comprise a substrate, wherein the method may comprise providing the 3D printable material to the substrate, thereby providing the 3D item comprising 3D printed material. Note that with such 3D printer, it is also possible to provide non-core-shell layers, such as by only extruding the 3D printable core material.

As indicated above, the method comprises depositing during a printing stage 3D printable material. Herein, the term “3D printable material” refers to the material to be deposited or printed, and the term “3D printed material” refers to the material that is obtained after deposition. These materials may be essentially the same, as the 3D printable material may especially refer to the material in a printer head or extruder at elevated temperature and the 3D printed material refers to the same material, but in a later stage when deposited. In embodiments, the 3D printable material may be printed as a filament and deposited as such. The 3D printable material may be provided as filament or may be formed into a filament. Hence, whatever starting materials are applied, a filament comprising 3D printable material may be provided by the printer head and 3D printed. The term “extrudate” may be used to define the 3D printable material downstream of the printer head, but not yet deposited. The latter may be indicated as “3D printed material”. In fact, the extrudate may be considered to comprises 3D printable material, as the material is not yet deposited. Upon deposition of the 3D printable material or extrudate, the material may thus be indicated as 3D printed material. Essentially, the materials may be the same material, as the thermoplastic material upstream of the printer head, downstream of the printer head, and when deposited, may essentially be the same material(s).

Herein, the term “3D printable material” may also be indicated as “printable material”. The term “polymeric material” may in embodiments refer to a blend of different polymers, but may in embodiments also refer to essentially a single polymer type with different polymer chain lengths. Hence, the terms “polymeric material” or “polymer” may refer to a single type of polymers but may also refer to a plurality of different polymers. The term “printable material” may refer to a single type of printable material but may also refer to a plurality of different printable materials. The term “printed material” may refer to a single type of printed material but may also refer to a plurality of different printed materials.

Hence, the term “3D printable material” may also refer to a combination of two or more materials. In general, these (polymeric) materials have a glass transition temperature T_{g} and/or a melting temperature Tₘ. The 3D printable material will be heated by the 3D printer before it leaves the nozzle to a temperature of at least the glass transition temperature, and in general at least the melting temperature. Hence, in a specific embodiment the 3D printable material comprises a thermoplastic polymer having a glass transition temperature (T_{g}) and/or a melting point (Tₘ), and the printer head action may comprise heating the 3D printable material above the glass transition and in embodiments above the melting temperature (especially when the thermoplastic polymer is a semi-crystalline polymer). In yet another embodiment, the 3D printable material comprises a (thermoplastic) polymer having a melting point (Tₘ), and the 3D printing stage may comprise heating the 3D printable material to be deposited on the receiver item to a temperature of at least the melting point. The glass transition temperature is in general not the same thing as the melting temperature. Melting is a transition which may occur in crystalline polymers. Melting may happen when the polymer chains fall out of their crystal structures, and become a disordered liquid. The glass transition may be a transition which happens to amorphous polymers; that is, polymers whose chains are not arranged in ordered crystals, but are just strewn around in any fashion, even though they are in the solid state. Polymers can be amorphous, essentially having a glass transition temperature and not a melting temperature or can be (semi) crystalline, in general having both a glass transition temperature and a melting temperature, with in general the latter being larger than the former. The glass temperature may e.g. be determined with differential scanning calorimetry. The melting point or melting temperature can also be determined with differential scanning calorimetry.

As indicated above, the invention may thus provide a method comprising providing a filament of 3D printable material and printing during a printing stage said 3D printable material on a substrate, to provide said 3D item.

Hence, in another aspect the invention provides a filament for producing a 3D item by means of fused deposition modelling. Especially, the filament may comprise 3D printable material. In embodiments, the 3D printable material may comprise one or more of a thermoplastic material, a photocatalytic material, and a pore forming material. Especially, the 3D printable material comprises a thermoplastic material, a photocatalytic material, and a pore forming material. Hence, in specific embodiments the invention provides a filament for producing a 3D item by means of fused deposition modelling, the filament comprising 3D printable material, wherein the 3D printable material comprises (i) a thermoplastic material, (ii) a photocatalytic material, and (iii) a pore forming material. However, in alternative specific embodiments, for instance for amongst others application in methods where the pore formation is induced by the introduction of a gas (see also above), the invention provides a filament for producing a 3D item by means of fused deposition modelling, the filament comprising 3D printable material, wherein the 3D printable material comprises (i) a thermoplastic material, and (ii) a photocatalytic material, and (substantially) no pore forming material.

As indicated above, in embodiments, the pore forming material may comprise a liquid at room temperature that boils at a temperature selected from the range of 30-500° C., such as 50-350° C., like 100-350° C., especially 150-300° C. Additionally or alternatively, the pore forming material may comprise a foaming agent. In embodiments, the pore forming material may comprise an inorganic foaming agent. In alternative embodiments, the pore forming material may comprise an organic foaming agent.

As indicated above, in embodiments, the particles may be spherical, cubic, prismoid, flakes, or irregularly shaped. Particle sizes are especially selected such that the particles can pass the printer nozzle without clog formation. Particles having a large surface area may be more efficient than spherical particles. Especially, particles may be flakes. Hence, the 3D printable material may in embodiments comprises flakes comprising the photocatalytic material. In embodiments, the flakes have flake dimensions defined by smallest rectangular prisms circumscribing the respective flakes, wherein such rectangular prism has a length L1, a width L2 and a height L3. In embodiments L1≥L2≥L3. In embodiments, length L1 is selected from the range of 50-2000 μm, especially selected from the range of 100-2000 μm, especially selected from the range of 250-1500 μm, more especially selected from the range of 500-1000 μm. Such rectangular prism has a first aspect ratio AR1=L1/L3, and a second aspect ratio AR2=L2/L3. In embodiments, the aspect ratios AR1 and AR2 are individually selected from the range of 1-10000, especially in the range of 2-1000, more especially in the range of 5-500, such as greater than 5. Hence, in specific embodiments, the pore forming material comprises a liquid at room temperature that boils at a temperature selected from the range of 100-350° C., wherein the 3D printable material comprises flakes comprising the photocatalytic material, wherein the flakes have flake dimensions defined by smallest rectangular prisms circumscribing the respective flakes, wherein such rectangular prism has a length L1, a width L2 and a height L3, wherein the length L1 is selected from the range of 50-2000 μm wherein a first aspect ratio is AR1=L1/L3, wherein a second aspect ratio is AR2=L2/L3, wherein the aspect ratios AR1 and AR2 are individually selected from the range of 1-10000. See further also above.

In this way, it may be possible to prepare high-performance photocatalytic 3D printed items by means of FDM, whilst starting from a previously prepared filament.

The pore forming material within the printable material may expand during heating. Therefore, the porosity of the filament may depend on the temperature used for the preparation of the filament. When the filament is prepared at a temperature lower than the printing temperature, the pores in the filament may be smaller than the pores in the 3D printed item or no pores may be present yet in the filament. In embodiments, the filament may be made at a first temperature T₁. Further, the 3D printing method may comprise 3D printing the filament, wherein the filament is heating in the printer nozzle at a second temperature T₂. In embodiments, T₂>T₁. Hence, in embodiments during printing porosity may be further increased. In embodiments T₂−T₁≥5° C., especially T₂−T₁≥10° C., more especially T₂−T₁≥20° C.

Alternatively, the filament may be prepared at the same temperature or a higher temperature than the printing, thus T₁≥T₂. Therefore, a density of the printed item nm may be the identical to or lower than a density of the filament n_{f}, thus nₘ≤n_{f}. Hence, nₘ/n_{f}≤1, especially nₘ/n_{f}≤0.9, such as nₘ/n_{f}≤0.7. However, in specific embodiments nₘ/n_{f}≥0.1, such as nₘ/n_{f}≥0.2, like nₘ/n_{f}≥0.25.

Materials that may especially qualify as 3D printable materials may be selected from the group consisting of metals, glasses, thermoplastic polymers, silicones, etc. Especially, the 3D printable material comprises a (thermoplastic) polymer selected from the group consisting of ABS (acrylonitrile butadiene styrene), Nylon (or polyamide), Acetate (or cellulose), PLA (poly lactic acid), terephthalate (such as PET polyethylene terephthalate), Acrylic (polymethylacrylate, Perspex, polymethylmethacrylate, PMMA), Polypropylene (or polypropene), Polycarbonate (PC), Polystyrene (PS), PE (such as expanded-high impact-Polythene (or polyethene), Low density (LDPE) High density (HDPE)), PVC (polyvinyl chloride) Polychloroethene, such as thermoplastic elastomer based on copolyester elastomers, polyurethane elastomers, polyamide elastomers polyolefine based elastomers, styrene based elastomers, etc. Optionally, the 3D printable material may comprise a 3D printable material selected from the group consisting of Urea formaldehyde, Polyester resin, Epoxy resin, Melamine formaldehyde, thermoplastic elastomer, etc. Optionally, the 3D printable material may comprise a 3D printable material selected from the group consisting of a polysulfone. Elastomers, especially thermoplastic elastomers, may especially be interesting as they are flexible and may help obtaining relatively more flexible filaments comprising the thermally conductive material. A thermoplastic elastomer may comprise one or more of styrenic block copolymers (TPS (TPE-s)), thermoplastic polyolefin elastomers (TPO (TPE-o)), thermoplastic vulcanizates (TPV (TPE-v or TPV)), thermoplastic polyurethanes (TPU (TPU)), thermoplastic copolyesters (TPC (TPE-E)), and thermoplastic polyamides (TPA (TPE-A)).

Suitable thermoplastic materials, such as also mentioned in WO2017/040893, may include one or more of polyacetals (e.g., polyoxyethylene and polyoxymethylene), poly(C₁₋₆ alkyl)acrylates, polyacrylamides, polyamides, (e.g., aliphatic polyamides, polyphthalamides, and polyaramides), polyamideimides, polyanhydrides, polyarylates, polyarylene ethers (e.g., polyphenylene ethers), polyarylene sulfides (e.g., polyphenylene sulfides), polyarylsulfones (e.g., polyphenylene sulfones), polybenzothiazoles, polybenzoxazoles, polycarbonates (including polycarbonate copolymers such as polycarbonate-siloxanes, polycarbonate-esters, and polycarbonate-ester-siloxanes), polyesters (e.g., polycarbonates, polyethylene terephthalates, polyethylene naphtholates, polybutylene terephthalates, polyarylates), and polyester copolymers such as polyester-ethers), polyetheretherketones, polyetherimides (including copolymers such as polyetherimide-siloxane copolymers), polyetherketoneketones, polyetherketones, polyethersulfones, polyimides (including copolymers such as polyimide-siloxane copolymers), poly(C₁₋₆ alkyl)methacrylates, polymethacrylamides, polynorbornenes (including copolymers containing norbornenyl units), polyolefins (e.g., polyethylenes, polypropylenes, polytetrafluoroethylenes, and their copolymers, for example ethylene-alpha-olefin copolymers), polyoxadiazoles, polyoxymethylenes, polyphthalides, polysilazanes, polysiloxanes, polystyrenes (including copolymers such as acrylonitrile-butadiene-styrene (ABS) and methyl methacrylate-butadiene-styrene (MBS)), polysulfides, polysulfonamides, polysulfonates, polysulfones, polythioesters, polytriazines, polyureas, polyurethanes, polyvinyl alcohols, polyvinyl esters, polyvinyl ethers, polyvinyl halides, polyvinyl ketones, polyvinyl thioethers, polyvinylidene fluorides, or the like, or a combination comprising at least one of the foregoing thermoplastic polymers. Embodiments of polyamides may include, but are not limited to, synthetic linear polyamides, e.g., Nylon-6,6; Nylon-6,9; Nylon-6,10; Nylon-6,12; Nylon-11; Nylon-12 and Nylon-4,6, preferably Nylon 6 and Nylon 6,6, or a combination comprising at least one of the foregoing. Polyurethanes that can be used include aliphatic, cycloaliphatic, aromatic, and polycyclic polyurethanes, including those described above. Also useful are poly(C₁₋₆ alkyl)acrylates and poly(C₁₋₆ alkyl)methacrylates, which include, for instance, polymers of methyl acrylate, ethyl acrylate, acrylamide, methacrylic acid, methyl methacrylate, n-butyl acrylate, and ethyl acrylate, etc. In embodiments, a polyolefine may include one or more of polyethylene, polypropylene, polybutylene, polymethylpentene (and co-polymers thereof), polynorbornene (and co-polymers thereof), poly 1-butene, poly(3-methylbutene), poly(4-methylpentene) and copolymers of ethylene with propylene, 1-butene, 1-hexene, 1-octene, 1-decene, 4-methyl-1-pentene and 1-octadecene.

In specific embodiments, the 3D printable material (and the 3D printed material) may comprise one or more of polycarbonate (PC), polyethylene (PE), high-density polyethylene (HDPE), polypropylene (PP), polyoxymethylene (POM), polyethylene naphthalate (PEN), styrene-acrylonitrile resin (SAN), polysulfone (PSU), polyphenylene sulfide (PPS), and semi-crystalline polytethylene terephthalate (PET), acrylonitrile butadiene styrene (ABS), poly(methyl methacrylate) (PMMA), polystyrene (PS), and styrene acrylic copolymers (SMMA).

The term 3D printable material is further also elucidated below, but may especially refer to a thermoplastic material, optionally including additives, to a volume percentage of at maximum about 60%, especially at maximum about 30 vol. %, such as at maximum 20 vol. % (of the additives relative to the total volume of the thermoplastic material and additives).

The printable material may thus in embodiments comprise two phases. The printable material may comprise a phase of printable polymeric material, especially thermoplastic material (see also below), which phase is especially an essentially continuous phase. In this continuous phase of thermoplastic material polymer additives such as one or more of antioxidant, heat stabilizer, light stabilizer, ultraviolet light stabilizer, ultraviolet light absorbing additive, near infrared light absorbing additive, infrared light absorbing additive, plasticizer, lubricant, release agent, antistatic agent, anti-fog agent, antimicrobial agent, colorant, laser marking additive, surface effect additive, radiation stabilizer, flame retardant, anti-drip agent may be present. The additive may have useful properties selected from optical properties, mechanical properties, electrical properties, thermal properties, and mechanical properties (see also above).

The printable material in embodiments may comprise particulate material, i.e. particles embedded in the printable polymeric material, which particles form a substantially discontinuous phase. The number of particles in the total mixture may especially not be larger than 60 vol. %, relative to the total volume of the printable material (including the (anisotropically conductive) particles) especially in applications for reducing thermal expansion coefficient. For optical and surface related effect number of particles in the total mixture may be equal to or less than 20 vol. %, such as up to 10 vol. %, relative to the total volume of the printable material (including the particles). Hence, the 3D printable material may especially refer to a continuous phase of essentially thermoplastic material, wherein other materials, such as particles, may be embedded. Likewise, the 3D printed material especially refers to a continuous phase of essentially thermoplastic material, wherein other materials, such as particles, are embedded. The particles may comprise one or more additives as defined above. Hence, in embodiments the 3D printable materials may comprises particulate additives.

When using a core-shell nozzle, the 3D printable material provided to the core of the core-shell nozzle may be a filament comprising 3D printable material or may be particulate 3D printable material. Both type of feeds may be extruded via the core of the core-shell nozzle. The 3D printable material provided to a shell of the core-shell nozzle may be particulate 3D printable material. Such particulate 3D printable material (feed) may be extruded via the shell of the core-shell nozzle. When using a nozzle with a single opening, the 3D printable material provided to nozzle may be a filament comprising 3D printable material or may be particulate 3D printable material. Both type of feeds may be extruded via the nozzle.

The printable material may be printed on a receiver item. Especially, the receiver item can be the building platform or can be comprised by the building platform. The receiver item can also be heated during 3D printing. However, the receiver item may also be cooled during 3D printing.

The phrase “printing on a receiver item” and similar phrases include amongst others directly printing on the receiver item, or printing on a coating on the receiver item, or printing on 3D printed material earlier printed on the receiver item. The term “receiver item” may refer to a printing platform, a print bed, a substrate, a support, a build plate, or a building platform, etc. Instead of the term “receiver item” also the term “substrate” may be used. The phrase “printing on a receiver item” and similar phrases include amongst others also printing on a separate substrate on or comprised by a printing platform, a print bed, a support, a build plate, or a building platform, etc. Therefore, the phrase “printing on a substrate” and similar phrases include amongst others directly printing on the substrate, or printing on a coating on the substrate or printing on 3D printed material earlier printed on the substrate. Here below, further the term substrate is used, which may refer to a printing platform, a print bed, a substrate, a support, a build plate, or a building platform, etc., or a separate substrate thereon or comprised thereby.

Layer by layer printable material may be deposited, by which the 3D printed item may be generated (during the printing stage). The 3D printed item may show a characteristic ribbed structures (originating from the deposited filaments). However, it may also be possible that after a printing stage, a further stage is executed, such as a finalization stage. This stage may include removing the printed item from the receiver item and/or one or more post processing actions. One or more post processing actions may be executed before removing the printed item from the receiver item and/or one more post processing actions may be executed after removing the printed item from the receiver item. Post processing may include e.g. one or more of polishing, coating, adding a functional component, etc. Post-processing may include smoothening the ribbed structures, which may lead to an essentially smooth surface.

Further, the invention relates to a software product that can be used to execute the method described herein. Therefore, in yet a further aspect the invention also provides a computer program product, when running on a computer which is functionally coupled to or comprised by a fused deposition modeling 3D printer, is capable of bringing about the method as described herein.

Hence, in an aspect the invention (thus) provides a software product, which, when running on a computer is capable of bringing about (one or more embodiments of) the method (for producing a 3D item by means of fused deposition modelling) as described herein.

The herein described method provides 3D printed items. Hence, the invention also provides in a further aspect a 3D printed item obtainable with the herein described method. In a further aspect a 3D printed item obtainable with the herein described method is provided.

Especially, the invention provides a 3D item comprising 3D printed material. As indicated above, the 3D item comprises a plurality of layers of 3D printed material. Especially one or more of the layers of 3D printed material may comprise photocatalytic material. Additionally, the 3D item may further comprise layers without photocatalytic material. Hence, especially at least part of the 3D printed material may comprise photocatalytic material. In embodiments, the 3D printed material may comprise photocatalytic material selected from the range of 0.5-20 wt %, like 1-15 wt %, such as 2-10 wt %. Especially one or more of the layers of 3D printed material may comprise pores. Additionally, the 3D item may further comprise layers without pores. In embodiments, at least part of the 3D printed material may have a pore volume selected from the range of 2-80 vol. %, like 5-70 vol. %, such as 10-60 vol. %, like 10-50 vol. %, especially 20-50 vol. %. Hence, in specific embodiments, the invention provides a 3D item comprising 3D printed material, wherein the 3D item comprises a plurality of layers of 3D printed material, wherein at least part of the 3D printed material comprises photocatalytic material selected from the range of 0.5-20 wt % and wherein at least part of the 3D printed material has a pore volume selected from the range of 10-50 vol. %. Hence, in embodiments the 3D printed material may comprise in the range of 0.5-20 wt % photocatalytic material. The weight percentage may especially refer to the total weight of the 3D printed material.

In embodiments, at least part of the 3D printed material may comprise particles comprising the photocatalytic material. As indicated above, in embodiments, the particles may be spherical, cubic, prismoid, flakes, or irregularly shaped. Particle sizes are especially selected such that the particles can pass the printer nozzle without clog formation. Particles having a large surface area may be more efficient than spherical particles. Especially, particles may be flakes. Hence, at least part of the 3D printed material may in embodiments comprises flakes comprising the photocatalytic material. In embodiments, the flakes have flake dimensions defined by smallest rectangular prisms circumscribing the respective flakes, wherein such rectangular prism has a length L1, a width L2 and a height L3. In embodiments L1≥L2≥L3. In embodiments, length L1 is selected from the range of 50-2000 μm, especially selected from the range of 100-2000 μm, especially selected from the range of 250-1500 μm, more especially selected from the range of 500-1000 μm. Such rectangular prism has a first aspect ratio AR1=L1/L3, and a second aspect ratio AR2=L2/L3. In embodiments, the aspect ratios AR1 and AR2 are individually selected from the range of 1-10000, especially in the range of 2-1000, more especially in the range of 5-500, such as greater than 5. Hence, in specific embodiments, at least part of the 3D printed material comprises flakes comprising the photocatalytic material, wherein the flakes have flake dimensions defined by smallest rectangular prisms circumscribing the respective flakes, wherein such rectangular prism has a length L1, a width L2 and a height L3, wherein the length L1 is selected from the range of 50-2000 μm, wherein a first aspect ratio is AR1=L1/L3, wherein a second aspect ratio is AR2=L2/L3, wherein the aspect ratios AR1 and AR2 are individually selected from the range of 1-10000, see further also above.

Especially, the 3D item comprises one or more layers of 3D printed material. More especially, the 3D item comprises a plurality of layers of 3D printed material. The 3D item may comprise two or more, like at least 5, such as at least 10, like in embodiments at least 20 layers of 3D printed material. The 3D printed item may especially comprise a plurality of layers on top of each other, i.e. stacked layers. The width (thickness) and height of (individually 3D printed) layers may e.g. in embodiments be selected from the range of 100-5000 μm, such as 200-2500 μm, with the height in general being smaller than the width. For instance, the ratio of height and width may be equal to or smaller than 0.8, such as equal to or smaller than 0.6.

Layers may be core-shell layers or may consist of a single material. Within a layer, there may also be a change in composition, for instance when a core-shell printing process was applied and during the printing process it was changed from printing a first material (and not printing a second material) to printing a second material (and not printing the first material).

At least part of the 3D printed item may include a coating.

Some specific embodiments in relation to the 3D printed item have already been elucidated above when discussing the method. Below, some specific embodiments in relation to the 3D printed item are discussed in more detail.

As indicated above, the photocatalytic particles may comprise TiO₂ particles.

In embodiments, the surface of the 3D item may be relatively smooth. Alternatively, the surface may have photocatalytic particles protruding into surrounding air. In this way, a larger contact surface area may result in more photocatalysis.

In embodiments, the thermoplastic material per se (i.e. without taking into account the photocatalytic particles) may be light transmissive, though this is not necessarily the case.

The transmission of the light transmissive material for one or more wavelengths (violet and/or UV) may be at least 80%/cm, such as at least 90%/cm, even more especially at least 95%/cm, such as at least 98%/cm, such as at least 99%/cm. This implies that e.g. a 1 cm3 cubic shaped piece of light transmissive material, under perpendicular irradiation of radiation having a selected wavelength in the visible, will have a transmission of at least 95%.

In embodiments, the 3D item may comprise one or more fluoropolymers. Embodiments of the fluoropolymers are described above.

As indicated above, photocatalytic material may desirably be present in higher concentrations near the outer surface of the layers. Therefore, in embodiments, the 3D item may comprise core-shell layers comprising (i) a core and (ii) a shell, wherein the shell at least partly encloses the core. Especially, the core and (ii) a shell may comprise thermoplastic material. In specific embodiments, a second concentration c2 of photocatalytic material comprised by the shell may be larger than a first concentration c1 of photocatalytic material in the core. In specific embodiments, c1/c2≤0.9, such as c1/c2≤0.8, like c1/c2≤0.5. In embodiments, no photocatalytic material may be present in the core. Thus in embodiments c1 may be zero.

In embodiments, where the pore forming material may have been a material that has been decomposed or has boiled, etc., see also above, the 3D printed material may have some residual material of the pore forming material.

The (with the herein described method) obtained 3D printed item may be functional per se. For instance, the 3D printed item may be a lens, a collimator, a reflector, etc. The thus obtained 3D item may (alternatively) be used for decorative or artistic purposes. The 3D printed item may include or be provided with a functional component. The functional component may especially be selected from the group consisting of an optical component, an electrical component, and a magnetic component. The term “optical component” especially refers to a component having an optical functionality, such as a lens, a mirror, a light transmissive element, an optical filter, etc. The term optical component may also refer to a light source (like a LED). The term “electrical component” may e.g. refer to an integrated circuit, PCB, a battery, a driver, but also a light source (as a light source may be considered an optical component and an electrical component), etc. The term magnetic component may e.g. refer to a magnetic connector, a coil, etc. Alternatively, or additionally, the functional component may comprise a thermal component (e.g. configured to cool or to heat an electrical component). Hence, the functional component may be configured to generate heat or to scavenge heat, etc. . . . .

As indicated above, the 3D printed item maybe used for different purposes. Amongst others, the 3D printed item maybe used in lighting. Hence, in yet a further aspect the invention also provides a lighting device comprising the 3D item as defined herein. In a specific aspect the invention provides a lighting system comprising (a) a light source configured to provide (visible) light source light and (b) the 3D item as defined herein, wherein 3D item may be configured as one or more of (i) at least part of a housing, (ii) at least part of a wall of a lighting chamber, and (iii) a functional component, wherein the functional component may be selected from the group consisting of an optical component, a support, an electrically insulating component, an electrically conductive component, a thermally insulating component, and a thermally conductive component. Hence, in specific embodiments the 3D item may be configured as one or more of (i) at least part of a lighting device housing, (ii) at least part of a wall of a lighting chamber, and (iii) an optical element. As a relative smooth surface may be provided, the 3D printed item may be used as mirror or lens, etc. In embodiments, the 3D item may be configured as shade. A device or system may comprise a plurality of different 3D printed items, having different functionalities.

The herein described method providing 3D printed items and the 3D items comprise high-performance photocatalytic layers. As indicated above, such high-performance photocatalytic layers may provide reactive oxygen species when irradiated with violet and/or UV light. Hence, the invention also provides in a further aspect a radiation generating system comprising the 3D item. Especially, the radiation generating system may in embodiments be (used as) a disinfection system. In embodiments, the radiation generating system may comprise the 3D item and a radiation generating device. Especially, the radiation generating device may be configured to generate device light comprising violet and/or UV light. In specific embodiments, the 3D item may be configured in a light receiving relationship with the light generating device. Hence, in embodiments the invention may provide a radiation generating system comprising the 3D item and a radiation generating device, wherein the radiation generating device is configured to generate device light comprising violet and/or UV light, and wherein the 3D item is configured in a light receiving relationship with the light generating device.

Additionally, the radiation generating system may in embodiments further comprise an air flow inducing device, such as a fan, blower or pump. The air flow inducing device may especially be configurated to promote flow of a gas along at least part of the 3D item. Hence, in embodiments the radiation generating system further comprises a fan to promote flow of a gas along at least part of the 3D item.

Such radiation generating system may be used for treating a gas. Hence, in embodiments, the invention also provides in a further aspect a method for treating a gas. In embodiments, the method may comprise contacting the gas with the 3D item from the radiation generating system. Especially, the method may comprise irradiating the 3D item with the radiation from the radiation generating system. Hence, the invention further provides a method for treating a gas, the method comprising contacting the gas with the 3D item from the radiation generating system and irradiating the 3D item with the radiation from the radiation generating system. Such method may be used for disinfection of the gas.

Returning to the 3D printing process, a specific 3D printer may be used to provide the 3D printed item described herein. Therefore, in yet a further aspect the invention also provides a fused deposition modeling 3D printer, comprising (a) a printer head comprising a printer nozzle, and (b) a 3D printable material providing device configured to provide 3D printable material to the printer head, wherein the fused deposition modeling 3D printer is configured to execute the method for 3D printing a 3D printed item as described herein.

The printer nozzle may include a single opening. In other embodiments, the printer nozzle may be of the core-shell type, having two (or more) openings. The term “printer head” may also refer to a plurality of (different) printer heads; hence, the term “printer nozzle” may also refer to a plurality of (different) printer nozzles.

The 3D printable material providing device may provide a filament comprising 3D printable material to the printer head or may provide the 3D printable material as such, with the printer head creating the filament comprising 3D printable material. Hence, in embodiments the invention provides a fused deposition modeling 3D printer, comprising (a) a printer head comprising a printer nozzle, and (b) a filament providing device configured to provide a filament comprising 3D printable material to the printer head, wherein the fused deposition modeling 3D printer is configured to provide said 3D printable material to a substrate, and to execute the method for 3D printing a 3D printed item as described herein. In embodiments, the 3D printer may comprise a gas entrance for introducing bubbles in the 3D printable material. Additionally or alternatively, a core nozzle of a core-shell printer may be used.

Especially, the 3D printer comprises a controller (or is functionally coupled to a controller) that is configured to execute in a controlling mode (or “operation mode”) the method as described herein. Instead of the term “controller” also the term “control system” (see e.g. above) may be applied.

The term “controlling” and similar terms especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc. Beyond that, the term “controlling” and similar terms may additionally include monitoring. Hence, the term “controlling” and similar terms may include imposing behavior on an element and also imposing behavior on an element and monitoring the element. The controlling of the element can be done with a control system, which may also be indicated as “controller”. The control system and the element may thus at least temporarily, or permanently, functionally be coupled. The element may comprise the control system. In embodiments, the control system and element may not be physically coupled. Control can be done via wired and/or wireless control. The term “control system” may also refer to a plurality of different control systems, which especially are functionally coupled, and of which e.g. one control system may be a master control system and one or more others may be slave control systems. A control system may comprise or may be functionally coupled to a user interface.

The control system may also be configured to receive and execute instructions form a remote control. In embodiments, the control system may be controlled via an App on a device, such as a portable device, like a Smartphone or I-phone, a tablet, etc. The device is thus not necessarily coupled to the lighting system, but may be (temporarily) functionally coupled to the lighting system.

Hence, in embodiments the control system may (also) be configured to be controlled by an App on a remote device. In such embodiments the control system of the lighting system may be a slave control system or control in a slave mode. For instance, the lighting system may be identifiable with a code, especially a unique code for the respective lighting system. The control system of the lighting system may be configured to be controlled by an external control system which has access to the lighting system on the basis of knowledge (input by a user interface of with an optical sensor (e.g. QR code reader) of the (unique) code. The lighting system may also comprise means for communicating with other systems or devices, such as on the basis of Bluetooth, WIFI, LiFi, ZigBee, BLE or WiMAX, or another wireless technology.

The system, or apparatus, or device may execute an action in a “mode” or “operation mode” or “mode of operation”. Likewise, in a method an action or stage, or step may be executed in a “mode” or “operation mode” or “mode of operation” or “operational mode”. The term “mode” may also be indicated as “controlling mode”. This does not exclude that the system, or apparatus, or device may also be adapted for providing another controlling mode, or a plurality of other controlling modes. Likewise, this may not exclude that before executing the mode and/or after executing the mode one or more other modes may be executed.

However, in embodiments a control system may be available, that is adapted to provide at least the controlling mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, that can only operate in a single operation mode (i.e. “on”, without further tunability).

Hence, in embodiments, the control system may control in dependence of one or more of an input signal of a user interface, a sensor signal (of a sensor), and a timer. The term “timer” may refer to a clock and/or a predetermined time scheme.

Instead of the term “fused deposition modeling (FDM) 3D printer” shortly the terms “3D printer”, “FDM printer” or “printer” may be used. The printer nozzle may also be indicated as “nozzle” or sometimes as “extruder nozzle”.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1c schematically depict some general aspects of the 3D printer and of an embodiment of 3D printed material;

FIGS. 2a-2b schematically depict some further aspects of the method and of the 3D printed material of the invention;

FIGS. 3a-3b schematically depict some further aspects of the method and of the 3D printed material of the invention;

FIGS. 4a-4b schematically depict some aspects of embodiments of particles;

FIG. 5 schematically depicts some aspects and embodiments; and

FIGS. 6a-6b schematically depicts applications.

The schematic drawings are not necessarily to scale.

### DETAILED DESCRIPTION OF THE EMBODIMENTS

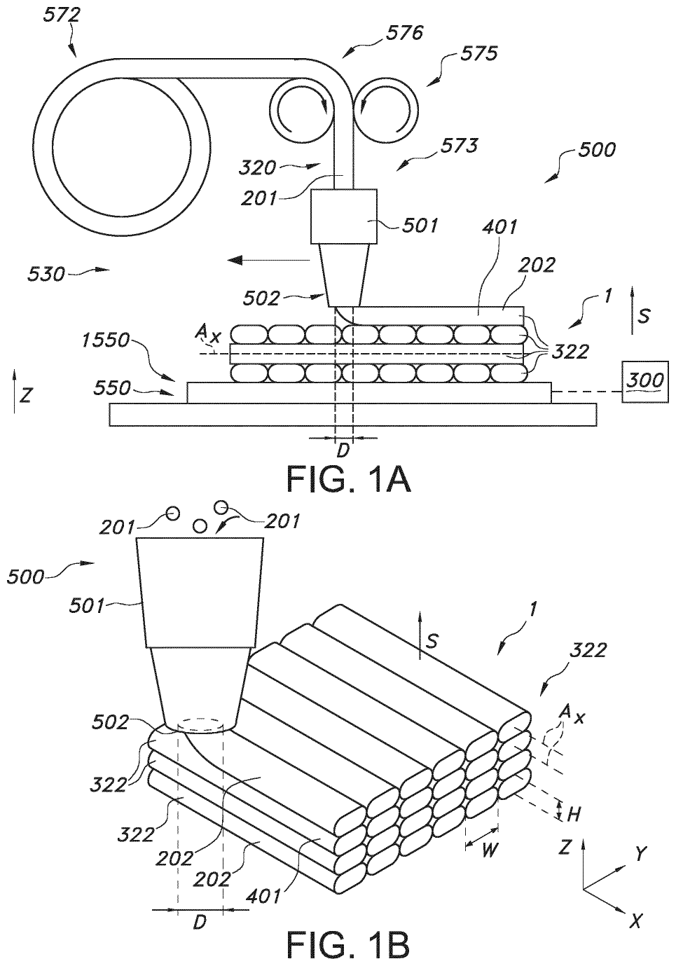

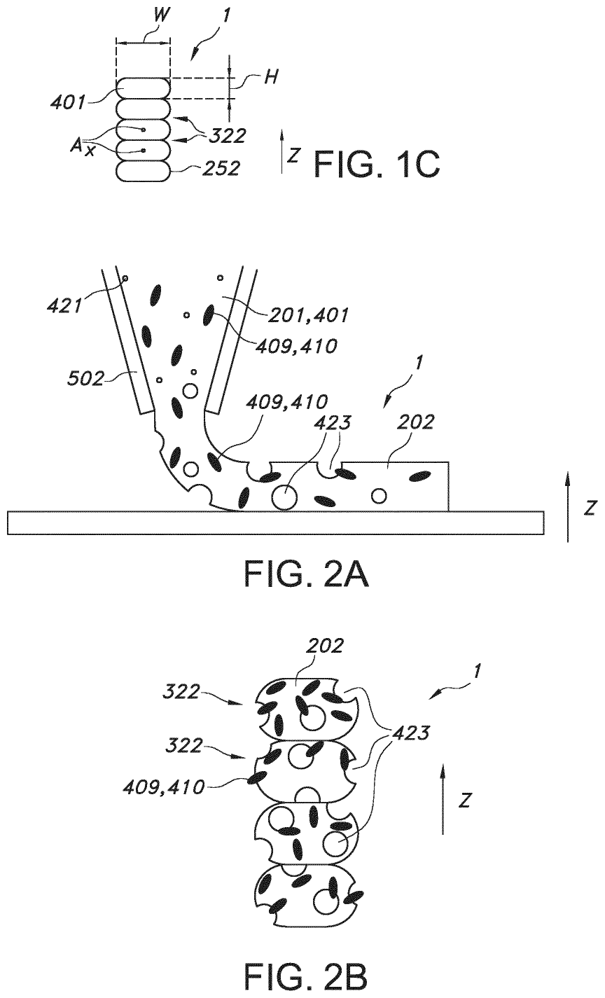

FIG. 1a schematically depicts some aspects of the 3D printer. Reference 500 indicates a 3D printer. Reference 530 indicates the functional unit configured to 3D print, especially FDM 3D printing; this reference may also indicate the 3D printing stage unit. Here, only the printer head for providing 3D printed material, such as an FDM 3D printer head is schematically depicted. Reference 501 indicates the printer head. The 3D printer of the present invention may especially include a plurality of printer heads (see below). Reference 502 indicates a printer nozzle. The 3D printer of the present invention may especially include a plurality of printer nozzles, though other embodiments are also possible. Reference 320 indicates a filament of printable 3D printable material (such as indicated above).

Instead of a filament also pellets may be used as 3D printable material. Both can be extruded via the printer nozzle.

For the sake of clarity, not all features of the 3D printer have been depicted, only those that are of especial relevance for the present invention (see further also below). Reference 321 indicates extrudate (of 3D printable material 201).

The 3D printer 500 is configured to generate a 3D item 1 by layer-wise depositing on a receiver item 550, which may in embodiments at least temporarily be cooled, a plurality of layers 322 wherein each layers 322 comprises 3D printable material 201, such as having a melting point Tₘ. The 3D printable material 201 may be deposited on a substrate 1550 (during the printing stage). By deposition, the 3D printable material 201 has become 3D printed material 202. 3D printable material 201 escaping from the nozzle 502 is also indicated as extrudate 321. Reference 401 indicates thermoplastic material.

The 3D printer 500 may be configured to heat the filament 320 material upstream of the printer nozzle 502. This may e.g. be done with a device comprising one or more of an extrusion and/or heating function. Such device is indicated with reference 573, and is arranged upstream from the printer nozzle 502 (i.e. in time before the filament material leaves the printer nozzle 502). The printer head 501 may (thus) include a liquefier or heater. Reference 201 indicates printable material. When deposited, this material is indicated as 3D printed material, which is indicated with reference 202. In embodiments, the 3D printable material 201 (and hence 3D printed material) may comprise one or more fluoropolymers.

Reference 572 indicates a spool or roller with material, especially in the form of a wire, which may be indicated as filament 320. The 3D printer 500 transforms this in an extrudate 321 downstream of the printer nozzle which becomes a layer 322 on the receiver item or on already deposited printed material. In general, the diameter of the extrudate 321 downstream of the nozzle 502 is reduced relative to the diameter of the filament 320 upstream of the printer head 501. Hence, the printer nozzle is sometimes (also) indicated as extruder nozzle. Arranging layer 322 by layer 322 and/or layer 322t on layer 322, a 3D item 1 may be formed. Reference 575 indicates the filament providing device, which here amongst others include the spool or roller and the driver wheels, indicated with reference 576.

Reference Ax indicates a longitudinal axis or filament axis.

Reference 300 schematically depicts a control system. The control system may be configured to control the 3D printer 500. The control system 300 may be comprised or functionally coupled to the 3D printer 500. The control system 300 may further comprise or be functionally coupled to a temperature control system configured to control the temperature of the receiver item 550 and/or of the printer head 501. Such temperature control system may include a heater which is able to heat the receiver item 550 to at least a temperature of 50° C., but especially up to a range of about 350° C., such as at least 200° C.

Alternatively or additionally, in embodiments the receiver plate may also be moveable in one or two directions in the x-y plane (horizontal plane). Further, alternatively or additionally, in embodiments the receiver plate may also be rotatable about z axis (vertical). Hence, the control system may move the receiver plate in one or more of the x-direction, y-direction, and z-direction.

Alternatively, the printer can have a head can also rotate during printing. Such a printer has an advantage that the printed material cannot rotate during printing.

Layers are indicated with reference 322, and have a layer height H and a layer width W.

Note that the 3D printable material is not necessarily provided as filament 320 to the printer head. Further, the filament 320 may also be produced in the 3D printer 500 from pieces of 3D printable material. Hence, the nozzle 502 may effectively produce from particulate 3D printable material 201 a filament 320, which upon deposition is indicated as layer 322 (comprising 3D printed material 202). Note that during printing the shape of the extrudate may further be changes, e.g. due to the nozzle smearing out the 3D printable material 201/3D printed material 202. FIG. 1b schematically depicts that also particulate 3D printable material 201 may be used as feed to the printer nozzle 502.

Reference D indicates the diameter of the nozzle (through which the 3D printable material 201 is forced). However, the nozzle is not necessarily circular.

FIG. 1b schematically depicts in 3D in more detail the printing of the 3D item 1 under construction. Here, in this schematic drawing the ends of the layers in a single plane are not interconnected, though in reality this may in embodiments be the case. Reference H indicates the height of a layer. Layers are indicated with reference 322. Here, the layers have an essentially circular cross-section. Often, however, they may be flattened, such as having an outer shape resembling a flat oval tube or flat oval duct (i.e. a circular shaped bar having a diameter that is compressed to have a smaller height than width, wherein the sides (defining the width) are (still) rounded).

Hence, FIG. 1a schematically depict some aspects of a fused deposition modeling 3D printer 500, comprising (a) a first printer head 501 comprising a printer nozzle 502, (b) a filament providing device 575 configured to provide a filament 320 comprising 3D printable material 201 to the first printer head 501, and optionally (c) a receiver item 550, which can be used to provide a layer of 3D printed material 202.

FIG. 1b schematically depict some aspects of a fused deposition modeling 3D printer 500 (or part thereof), comprising a first printer head 501 comprising a printer nozzle 502, and optionally a receiver item (not depicted), which can be used to which can be used to provide a layer of 3D printed material 202. Such fused deposition modeling 3D printer 500 may further comprise a 3D printable material providing device, configured to provide the 3D printable material 201 to the first printer head.

In FIGS. 1a-1b, the first or second printable material or the first or second printed material are indicated with the general indications printable material 201 and printed material 202, respectively. Downstream of the nozzle 502, the filament 320 with 3D printable material becomes, when deposited, layer 322 with 3D printed material 202. In FIG. 1b, by way of example the extrudate is essentially directly the layer 322 of 3D printed material 202, due to the short distance between the nozzle 502 and the 3D printed material (or receiver item (not depicted).

FIG. 1c schematically depicts a stack of 3D printed layers 322, each having a layer height H and a layer width W. Note that in embodiments the layer width and/or layer height may differ for two or more layers 322. The layer width and/or layer height may also vary within a layer. Reference 252 in FIG. 1c indicates the item surface of the 3D item (schematically depicted in FIG. 1c).

Referring to FIGS. 1a-1c, the filament of 3D printable material that is deposited leads to a layer having a height H (and width W). Depositing layer 322 after layer 322, the 3D item 1 is generated. FIG. 1c very schematically depicts a single-walled 3D item 1.

FIG. 2a schematically depicts further embodiments of the invention. Especially, the printable material 201 may comprise a thermoplastic material 401 and a photocatalytic material 409. In embodiments, the printable material 201 may further comprise a pore forming material 421. Especially, during at least part of the 3D printing stage the method may comprise producing pores 423 in the 3D printable material 201. In specific embodiments, during at least part of the 3D printing stage the method may comprise producing pores 423 by conversion of the pore forming material 421. Especially, the pore forming material 421 may comprise a material having a boiling point T_{b}. In embodiments, the 3D printing stage may comprise heating the pore forming material 421 in the printer nozzle 502. Especially, the printer nozzle 502 may have a nozzle temperature Tₙ, wherein 50° C.≤T_{b}≤Tₙ. In specific embodiments, the pore forming material 421 comprises a liquid at room temperature that boils at a temperature selected from the range of 100-350° C. Additionally or alternatively, the pore forming material may comprise a foaming agent. Especially, the 3D printing stage may comprise selecting the pore forming material 421, the 3D printable material 201, and the 3D printing conditions such that the 3D printed material 202 has a pore volume selected from the range of 10-50 vol. %.

FIG. 2b schematically depicts a stack of 3D printed layers 322 comprising pores 423 and photocatalytic material 409. Such item 1 may be obtained by the method of this invention. Especially, the 3D item 1 comprises a plurality of layers 322 of 3D printed material 202. In embodiments, at least part of the 3D printed material 202 may comprise photocatalytic material 409. Especially, at least part of the 3D printed material 202 may have a pore volume selected from the range of 10-50 vol. %. In the depicted embodiment, at least part of the 3D printed material 202 comprises flakes 410 comprising the photocatalytic material 409. Further embodiments of the flakes 410 are discussed in more detail below.

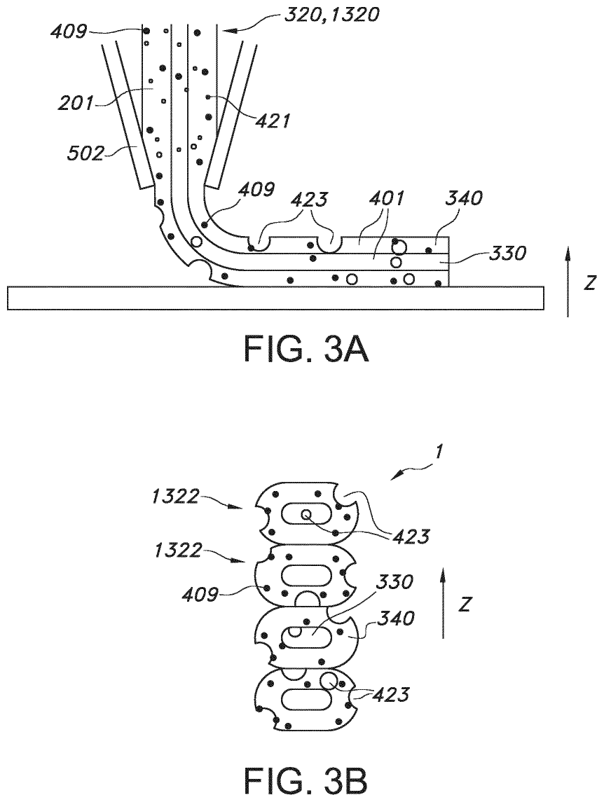

FIG. 3a depicts a further embodiment of the method, schematically illustrating using a filament 320. In embodiments, the 3D printing stage comprises: layer-wise depositing a filament 320 comprising the 3D printable material 201. Especially, the filament 320 may comprise a core-shell filament 1320 comprising (i) a core 330 and (ii) a shell 340, wherein the shell 340 at least partly encloses the core 330. Especially, the core 330 and (ii) the shell 340 comprise thermoplastic material 401. In specific embodiments, a second concentration of photocatalytic material 409 comprised by the shell 340 is larger than a first concentration of photocatalytic material in the core 330. FIG. 3b schematically depicts an embodiment of the 3D item 1 obtainable by such method. The depicted item 1 comprises a stack of core-shell layers 1322 comprising a core 330 and a shell 340. Especially, the 3D printed material 202 may comprise thermoplastic material 401. In specific embodiments, a second concentration of photocatalytic material 409 comprised by the shell 340 is larger than a first concentration of photocatalytic material in the core 330. Additionally or alternatively, the shell may in embodiments comprise more pores 423 than the core. However, the filament 320 does herein not necessarily comprise a core-shell filament 1320.

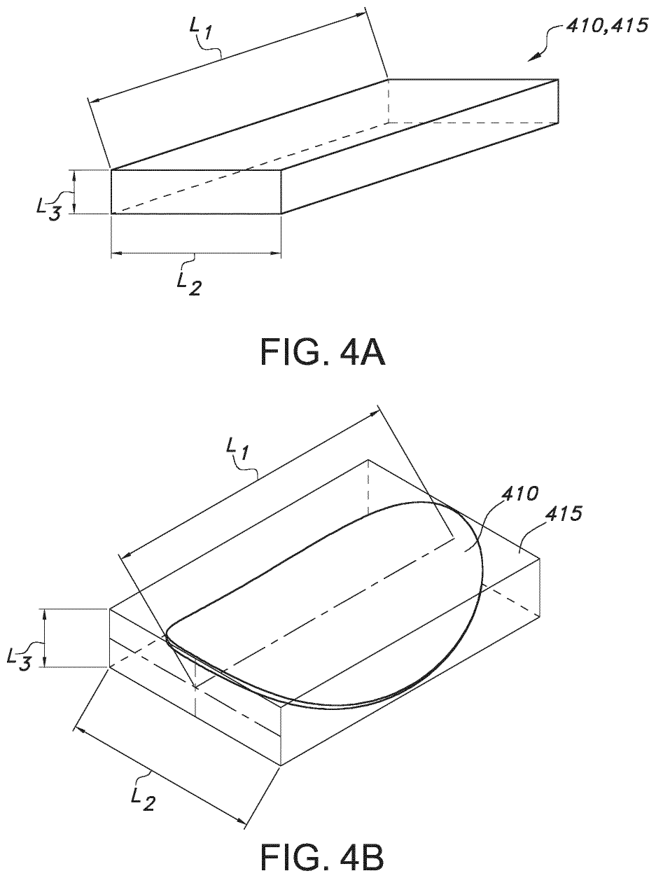

FIGS. 4a-4b are especially used to describe size of particles have not highly symmetrical shapes, like cubic or spherical, but such as flakes. FIGS. 4a-4b schematically depict embodiments of (photocatalytic) particles or flakes 410. FIG. 4a depicts a flake 410 that has a rectangular prism shape, wherein the rectangular prism 415 has a length L1, a width L2 and a height L3 wherein L1≥L2≥L3. FIG. 4b schematically depicts a flake that has a less regular shape such as pieces of broken glass, with a virtual smallest rectangular prism 415 enclosing the particle. The rectangular prism 415 has a length L1, a width L2 and a height L3 wherein L1≥L2≥L3. The rectangular prism 415 has a first aspect ratio AR1=L1/L3 and a second aspect ratio is AR2=L2/L3.

Further, note that the flakes are not essentially oval or rectangular prismoids. The flakes may have any shape, especially wherein length L1 is selected from the range of 50-2000 μm and the aspect ratios are in the range of 1-10000. Of course, the flakes may comprise a combination of differently shaped particles.

Also, the particulate material that is embedded in the 3D printable material or is embedded in the 3D printed material may include a broad distribution of particles sizes.

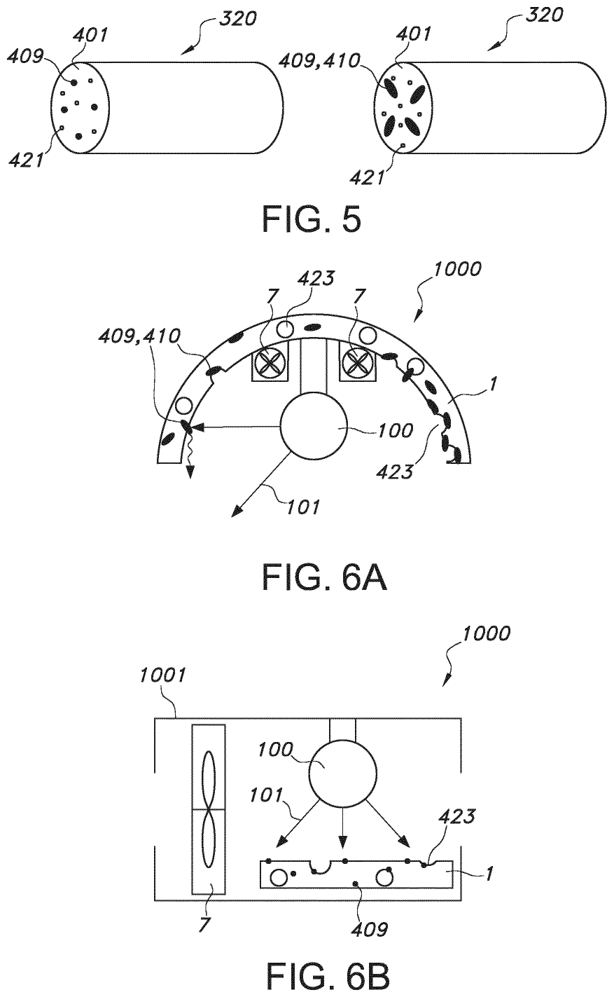

FIG. 5 depicts some embodiments of a filament 320 that may be used in the method. The filaments 320 may be used in a printer 500, e.g. as depicted in FIG. 1a-1b, having a nozzle 502 with a single opening. Especially, the filament 320 may comprise 3D printable material 201, wherein the 3D printable material 201 comprises (i) a thermoplastic material 401, (ii) a photocatalytic material 409, and (iii) a pore forming material 421. In specific embodiments, the pore forming material 421 comprises a liquid at room temperature that boils at a temperature selected from the range of 100-350° C. Additionally or alternatively, the pore forming material may comprise a foaming agent. Especially, the 3D printable material 201 may comprise flakes 410 comprising the photocatalytic material 409. The flakes 410 are described in more detail above.

FIG. 6a schematically depicts embodiments of a radiation generating system 1000 comprising the 3D item 1 and a radiation generating device 100. In the depicted embodiments, the radiation generating device 100 is configured to generate device light 101 comprising violet and/or UV light. Especially, the 3D item 1 (comprising photocatalytic material 409 such as flakes comprising the photocatalytic material 410) may be configured in a light receiving relationship with the light generating device 100. The light generating device 100 may (during operation) generate device light 101. Especially, the device light 101 may comprise violet light and/or UV light. As depicted, the device light 101 may be converted into reactive oxygen species by the photocatalytic material 409. In specific embodiments, the radiation generating system 1000 may further comprise a fan 7 to promote flow of a gas along at least part of the 3D item 1. In this way, the formed reactive oxygen species may be transported (further) away from the 3D item 1. The depicted applications may be used in a method for treating a gas. Especially, the method may comprise contacting the gas with the 3D item 1 from the radiation generating system 1000 and irradiating the 3D item 1 with the radiation 101 from the radiation generating system 1000 as depicted. The radiation generating system may in embodiments be or comprise a lamp or luminaire as depicted in FIG. 6a. The lamp may comprise a housing or shade or another element, which may comprise or be the 3D printed item 1. Here, the half sphere (in cross-sectional view) schematically indicates a housing or shade. Here, the device light 101 may in embodiments further comprise visible light.

In alternative embodiments, the radiation generating system 1000 may be a more closed system, such as depicted in FIG. 6b. In the depicted embodiment, the radiation generating system 1000 may comprise a housing 1001. Especially such radiation generating system 1000 may comprise a flow generating device such as a fan 7 to promote the produced reactive oxygen species to exit the housing 1001.

In yet other embodiments, the radiation generating system 1000 may be comprised by a light generating system, such as a luminaire, or a light generating system comprising a luminaire.

The term “plurality” refers to two or more. The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/of”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. The article “a” or “an” preceding an element does not exclude the presence of a plurality of such elements.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

It goes without saying that one or more of the first (printable or printed) material and second (printable or printed) material may contain fillers such as glass and fibers which do not have (to have) influence on the on T_{g} or Tₘ of the material(s).

1. A method for producing a 3D item by means of fused deposition modelling, the method comprising: a 3D printing stage comprising layer-wise depositing 3D printable material, to provide the 3D item comprising 3D printed material, wherein the 3D item comprises layers of 3D printed material, wherein the 3D printable material comprises a thermoplastic material and a photocatalytic material wherein during at least part of the 3D printing stage the method comprises producing pores in the 3D printable material.

2. The method according to claim 1, wherein the 3D printable material further comprises a pore forming material, wherein during at least part of the 3D printing stage the method comprises producing pores by conversion of the pore forming material.

3. The method according to claim 2, comprising: using a 3D printing apparatus, wherein the 3D printing apparatus comprises a printer nozzle, wherein the pore forming material comprises a material having a boiling point T_{b}, wherein the 3D printing stage comprises heating the pore forming material in the printer nozzle wherein the printer nozzle has a nozzle temperature Tₐ, wherein 50° C.≤T_{b}≤Tₙ.

4. The method according to claim 2, wherein the pore forming material comprises one or more of (i) a liquid at room temperature that boils at a temperature selected from the range of 75-350° C. and (ii) a foaming agent.

5. The method according to claim 2, wherein the 3D printing stage comprises selecting the pore forming material, the 3D printable material, and the 3D printing conditions such that the 3D printed material has a pore volume selected from the range of 10-50 vol. %.

6. The method according to claim 1, wherein the 3D printable material comprises flakes comprising the photocatalytic material, wherein the flakes have flake dimensions defined by smallest rectangular prisms circumscribing the respective flakes, wherein such rectangular prism has a length (L1), a width (L2), and a height (L3), wherein the length (L1) is selected from the range of 50-2000 μm, wherein a first aspect ratio is AR1=L1/L3, wherein a second aspect ratio is AR2=L2/L3, wherein the aspect ratios AR1 and AR2 are individually selected from the range of 1-10000.

7. The method according to claim 1, wherein the 3D printable material comprises one or more fluoropolymers.

8. The method according to claim 1, wherein the 3D printing stage comprises: layer-wise depositing a filament comprising the 3D printable material, wherein the filament comprises a core-shell filament comprising (i) a core and (ii) a shell, wherein the shell at least partly encloses the core, wherein the core and (ii) a shell comprise thermoplastic material; wherein a second concentration of photocatalytic material comprised by the shell is larger than a first concentration of photocatalytic material in the core.

9. A filament for producing a 3D item by means of fused deposition modelling, the filament comprising 3D printable material, wherein the 3D printable material comprises (i) a thermoplastic material, (ii) a photocatalytic material, and (iii) a pore forming material.

10. The filament according to claim 9, wherein the pore forming material comprises a liquid at room temperature that boils at a temperature selected from the range of 100-350° C., wherein the 3D printable material comprises flakes comprising the photocatalytic material, wherein the flakes have flake dimensions defined by smallest rectangular prisms circumscribing the respective flakes, wherein such rectangular prism has a length (L1), a width (L2), and a height (L3), wherein the length (L1) is selected from the range of 50-2000 μm, wherein a first aspect ratio is AR1=L1/L3, wherein a second aspect ratio is AR2=L2/L3, wherein the aspect ratios AR1 and AR2 are individually selected from the range of 1-10000.

11. A 3D item comprising 3D printed material, wherein the 3D item comprises a plurality of layers of 3D printed material, wherein at least part of the 3D printed material has a pore volume selected from the range of 10-50 vol. %, and wherein the 3D printed material comprises 0.5-20 wt % of the photocatalytic material.

12. The 3D item according to claim 11, wherein at least part of the 3D printed material comprises flakes comprising the photocatalytic material, wherein the flakes have flake dimensions defined by smallest rectangular prisms circumscribing the respective flakes, wherein such rectangular prism has a length (L1), a width (L2), and a height (L3), wherein the length (L1) is selected from the range of 50-2000 μm, wherein a first aspect ratio is AR1=L1/L3, wherein a second aspect ratio is AR2=L2/L3, wherein the aspect ratios AR1 and AR2 are individually selected from the range of 1-10000; and wherein the 3D printed material comprises in the range of 0.5-20 wt % photocatalytic material.

13. A radiation generating system comprising (i) the 3D item according to claim 11, and (ii) a radiation generating device, wherein the radiation generating device is configured to generate device light comprising violet and/or UV light, and wherein the 3D item is configured in a light receiving relationship with the light generating device.

14. The radiation generating system according to claim 13, wherein the radiation generating system further comprises a fan to promote flow of a gas along at least part of the 3D item.

15. A method for treating a gas, the method comprising contacting the gas with the 3D item from the radiation generating system) according to claim 13 and irradiating the 3D item with the device light from the radiation generating system.
